(19) United States
(12) Patent Application Publication
(10) Pub. No.: US 2024/0207545 A1
(43) Pub. Date: Jun. 27, 2024

(54) NOISE REDUCTION BOX AND RESPIRATOR

(71) Applicant: BMC (TIANJIN) MEDICAL CO., LTD., Tianjin (CN)

(72) Inventors: Yunjing CHEN, Tianjin (CN); Qingsong WANG, Tianjin (CN); Xin TIAN, Tianjin (CN); Zhi ZHUANG, Tianjin (CN)

(21) Appl. No.: 18/394,342

(22) Filed: Dec. 22, 2023

(30) Foreign Application Priority Data
Dec. 23, 2022 (CN) 202223469940.4

(51) Int. Cl. A61M 16/00; G10K 11/178

(52) U.S. Cl. CPC A61M 16/0003; A61M 16/0066; G10K 11/17823; G10K 11/17873; A61M 2205/3375; A61M 2205/42; G10K 2210/116; G10K 2210/3011; G10K 2210/3027; G10K 2210/3044

(57) ABSTRACT

Disclosed are a noise reduction box and a respirator. The noise reduction box includes a housing and a blower. The housing includes a first chamber, a second chamber, an air inlet and an air outlet. The first chamber and the second chamber are connected to each other, and the air inlet and the air outlet are connected to the first chamber respectively. The blower is disposed in the first chamber, and includes an air intake connected to the second chamber and a tuyere connected to the air outlet.

### CROSS REFERENCE TO THE RELATED APPLICATIONS

This application claims priority to Chinese Patent Application No. 202223469940.4, filed on Dec. 23, 2022, the entire disclosure of which is incorporated herein by reference.

### TECHNICAL FIELD

The present application relates to the technical field of medical equipment, in particular to a noise reduction box and a respirator.

### BACKGROUND

In modern clinical medicine, respirators are used as an effective means that can artificially replace the function of spontaneous ventilation, and are widely used in anesthesia respiratory management, respiratory support therapy and first-aid and resuscitation. The respirator extracts air from a blower, and then supplies the air to the user through a humidifier. Since the respirator is generally required to be placed next to the user, and a loud noise is generated when the gas is sucked by the blower and flows inside the machine, the user's sleep will be seriously affected.

A noise reduction box is generally disposed inside the respirator so as to reduce the noise generated during the use of the respirator. Currently, two types of noise reduction boxes, sound-absorbing cotton type and expansion chamber type, are commonly used on the market. However, both of these two types of noise reduction boxes have different defects. Due to the material properties of the sound-absorbing cotton, a noise reduction box of the sound-absorbing cotton type is prone to produce fine particles after long-term use. Once these fine particles are inhaled by the patient, they will cause health risks to the patient. Since a noise reduction box of the expansion chamber type relies on multiple expansion chambers for noise reduction, an overall volume of the noise reduction box is large, which is not conducive to the structural design of the respirator.

### SUMMARY

In view of the above, the present application provides a noise reduction box and a respirator to at least solve the problems of existing noise reduction boxes that the patient's health is threatened during the noise reduction, or that the noise reduction box occupies a large space.

To achieve the above objects, the technical solution of the present disclosure is implemented as follows:

The present disclosure provides a noise reduction box, including a housing and a blower:
the housing includes a first chamber, a second chamber, an air inlet and an air outlet, the first chamber and the second chamber are connected to each other, and the air inlet and the air outlet are connected to the first chamber respectively;
the blower is disposed in the first chamber, and includes an air intake connected to the second chamber and a tuyere connected to the air outlet.

In some embodiments, an accommodation space of the first chamber is greater than the accommodation space of the second chamber.

In some embodiments, the noise reduction box further includes a partition; the partition plate is disposed between the first chamber and the second chamber to separate the first chamber and the second chamber;

a through hole is provided on the partition plate, and the first chamber is communicated with the second chamber via the through hole.

In some embodiments, in a direction perpendicular to the partition plate, a projection of the through hole is located on a periphery of the projection of the air intake.

In some embodiments, a flexible casing is sheathed around the blower.

In some embodiments, the noise reduction box further includes a speaker;
wherein the speaker is fixedly installed on the housing, and the speaker is used for generating a sound wave to counteract a noise wave of the blower.

In some embodiments, the noise reduction box further includes a control chip;
wherein the control chip is fixedly installed on the housing and is electrically connected to the speaker; the control chip is configured to control the speaker to generate the sound wave.

In some embodiments, the noise reduction box further includes a microphone;
wherein the microphone is fixedly installed on the housing, and is electrically connected to the control chip;
the microphone is configured to capture a frequency of the noise of the blower, and transmit the frequency of the noise to the control chip.

In some embodiments, the speaker and the microphone are disposed oppositely on both sides of the first chamber.

### BRIEF DESCRIPTION OF THE DRAWINGS

The drawings that constitute a part of the present disclosure are used to provide a further understanding of the present disclosure. The schematic embodiments of the present disclosure and their illustrations are used to explain the present disclosure, and do not constitute an improper limitation of the present disclosure. In the accompanying drawings:

FIG. 1 is a schematic appearance diagram of a noise reduction box according to an embodiment;

FIG. 2 is an exploded structural diagram of a noise reduction box according to an embodiment;

FIG. 3 is a cross-sectional view of a noise reduction box according to an embodiment along a direction perpendicular to a partition plate;

FIG. 4 is a cross-sectional view of a noise reduction box according to an embodiment along a direction parallel to a partition plate;

FIG. 5 is a top view of a noise reduction box according to an embodiment;

FIG. 6 is a bottom view of a noise reduction box according to an embodiment;

FIG. 7 is a side view of a noise reduction box according to an embodiment; and

FIG. 8 is a side view of another noise reduction box according to an embodiment.

### REFERENCE SIGN LIST

1—Housing; 101—Top cover; 102—Back case; 11—First chamber; 12—Second chamber; 13—Air inlet; 14—Air outlet; 2—Blower; 21—Air intake; 22—Tuyere; 23—Protrusion; 24—Flexible casing; 3—Partition plate; 31—Through hole; 4—Control chip; 5—Speaker; 6—Microphone.

### DETAILED DESCRIPTION OF THE EMBODIMENTS

The technical solutions of embodiments of the present disclosure will be clearly and thoroughly described below with reference to accompanying drawings of the embodiments. Apparently, the embodiments described are merely some embodiments rather than all the embodiments of the present disclosure. Based on the embodiments of the present disclosure, those skilled in the art may obtain all other embodiments without paying any creative effort, and all the embodiments fall within the scope of the protection of the present application.

The terms “first”, “second” and the like as used in the description and claims of the present disclosure are used to distinguish similar objects, and are not used to describe a specific order or sequence. It is to be understood that the data as indicated by such terms may be interchangeable under appropriate circumstances so that embodiments of the present disclosure may be implemented in sequences other than those illustrated or described herein, and objects as distinguished by terms such as “first”, “second” usually belong to one type, and the number of the objects is not limited. For example, the first object may include one object or multiple objects. In addition, “and/or” as used in the description and claims indicates at least one of the objects connected by “and/or”, and the character “/” generally indicates that the related objects are in an “or” relationship.

It should be understood that reference throughout this specification to “an embodiment” means that a particular feature, structure, or characteristic associated with the embodiment is included in at least one embodiment of the present disclosure. Thus, the phrase “in one embodiment” throughout the specification is not necessarily referring to the same embodiment. In addition, the particular features, structures or characteristics may be combined in any suitable 5 manner in one or more embodiments.

The noise reduction box and respirator provided by the present disclosure will be introduced in detail below by enumerating specific embodiments.

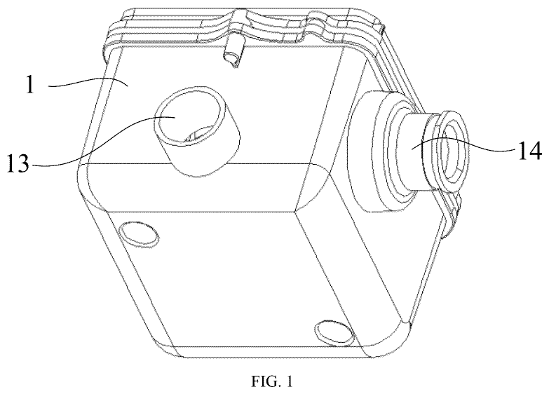

Referring to FIG. 1 to FIG. 4, the noise reduction box provided by the present disclosure includes a housing 1 and a blower 2. The housing 1 includes a first chamber 11, a second chamber 12, an air inlet 13 and an air outlet 14. The first chamber 11 and the second chamber 12 are connected to each other, and the air inlet 13 and the air outlet 14 are respectively connected to the first chamber 11. The blower 2 is disposed in the first chamber 11, and includes an air intake 21 connected to the second chamber 12 and a tuyere 22 connected to the air outlet 14.

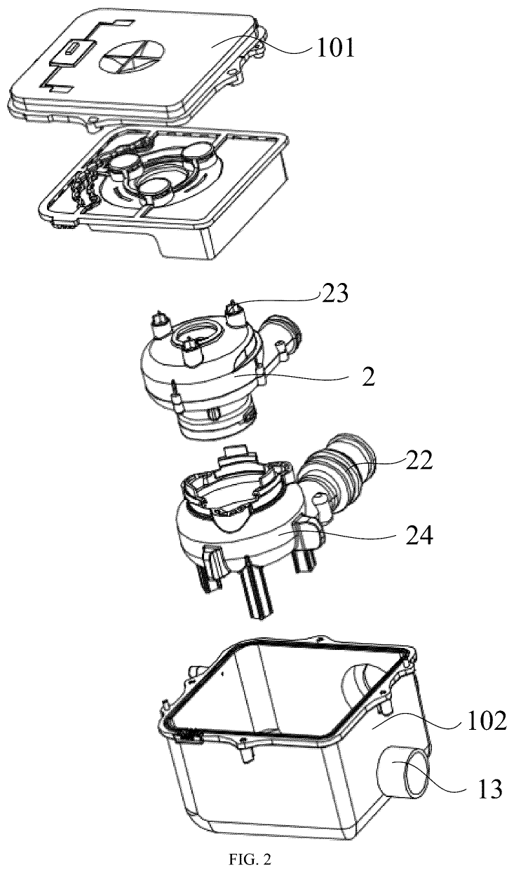

Specifically, as shown in FIG. 1 and FIG. 2, the noise reduction box includes the housing 1 and the blower 2, and the housing 1 is composed of a top cover 101 and a back case 102. The top cover 101 is used for seal-capping the back case 102 to seal the housing 1. The top cover 101 and the back case 102 may be made from materials such as hard alloy, plastic and plasthetics. Those skilled in the art may reasonably select materials based on processing costs and processing techniques, which is not limited in the embodiment. Preferably, the top cover 101 and the back case 102 of this embodiment are made from plastic, which has a good strength and can shield noises.

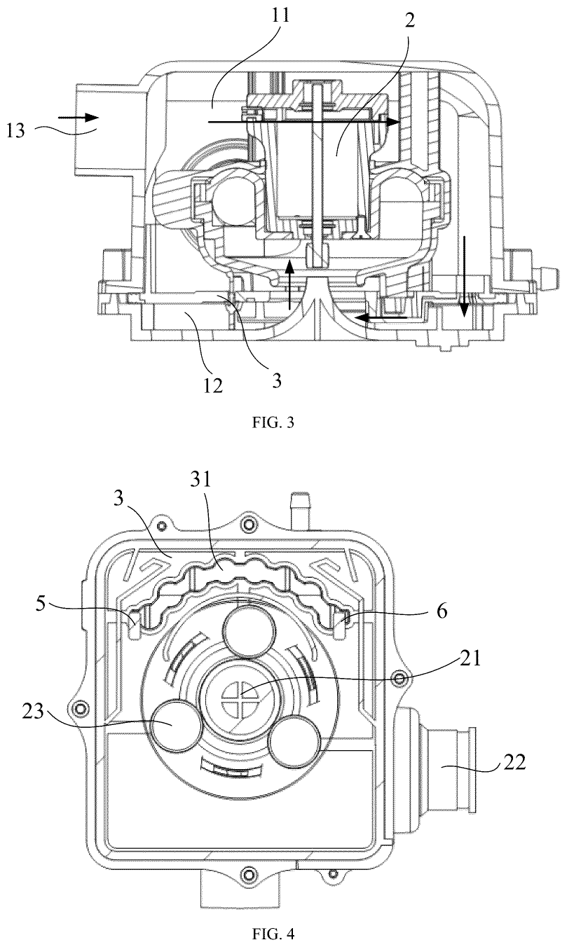

As shown in FIG. 1 and FIG. 3, the housing 1 includes the first chamber 11, the second chamber 12, the air inlet 13 and the air outlet 14. The first chamber 11 and the second chamber 12 are connected to each other, and the air inlet 13 and the air outlet 14 are connected to the first chamber 11 respectively. The air from an external environment enters the first chamber 11 through the air inlet 13, and is discharged from the first chamber 11 through the air outlet 14. Since the first chamber 11 and the second chamber 12 are connected to each other, the ventilation inside the housing 1 may be achieved through the air inlet 13 and the air outlet 14.

As shown in FIG. 1 and FIG. 4, the blower 2 is installed on the back case 102, and is arranged in the first chamber 11. The blower 2 includes an air intake 21 and a tuyere 22. The blower 2 has a relatively high rotational speed, and can suck air in through the air intake 21 and blow out the air through the tuyere 22. The air intake 21 is connected to the second chamber 12, and the tuyere 22 is connected to the air outlet 14, that is, the blower 2 sucks air from the second chamber 12 and blows the air out from the first chamber 11.

During the use of the noise reduction box, a flowing direction of the air in the noise reduction box is indicated by arrows in FIG. 3. The air in the external environment enters the first chamber 11 in the housing 1 through the air inlet 13, and performs cooling and heat dissipation on the blower 2 located in the first chamber 11, so that the blower 2 always runs in a relatively ideal temperature environment. Therefore, the blower 2 is prevented from malfunction due to overheating. Moreover, the air in the first chamber 11 continues to flow and diffuse to the second chamber 12, then the air is guided by the second chamber 12 to smoothly enter the air intake 21 of the blower 2, thereby reducing the noise generated when the air is inhaled by the blower 2. In a preferred embodiment, a protrusion 23 may be provided on the periphery of the air intake 21. The protrusion 23 may be a conical protrusion, a cylindrical protrusion, a frustum cone-shaped protrusion, etc., which is not limited in this embodiment. Since the protrusion 23 has a curved surface or inclined surface structure, through which the air may be guided to enter the air intake 21 smoothly, thereby further reducing the noise generated during the air intake process. In this embodiment, both the first chamber 11 and the second chamber 12 have a blocking effect on the noise generated by the blower 2, so that a part of the sound energy of the noise is converted into heat energy, and discharged out of the chamber along with the air. Accordingly, the function of reducing noise is achieved.

The noise reduction box of this embodiment adopts the first chamber 11 and the second chamber 12 to block part of the noise generated during the operation of the blower 2. Moreover, the first chamber 11 may cool or dissipate heat from the blower 2, and the second chamber 12 may guide the air to smoothly enter the air intake 21 of the blower 2, thereby further reducing the noise generated when the air is inhaled by the blower 2, and achieving the noise reduction function of the noise reduction box. Since the noise reduction box abandons the traditional sound-absorbing cotton structural design, the risk that fine particles produced during the noise reduction process of the noise reduction box are easily inhaled by the patient is eliminated. Moreover, since the traditional expansion cavity design is canceled, the volume of the noise reduction box is effectively reduced, thereby reducing the space occupied by the box, and facilitating the transportation and assembly of the noise reduction box.

In some embodiments, referring to FIG. 3, the accommodation space of the first chamber 11 is greater than the accommodation space of the second chamber 12.

Specifically, since the blower 2 is disposed in the first chamber 11 and the air in the first chamber 11 is used for cooling and dissipating heat from the blower 2, the first chamber 11 has a larger accommodation space and can accommodate more air. In this way, the blower 2 is ensured to operate in a suitable temperature environment, and the overheating of the blower 2 is avoided, thereby ensuring the wind speed at the air inlet and air outlet of the blower 2, and improving the heat dissipation effect while reducing noise. Moreover, the accommodation space of the first chamber 11 is greater than the accommodation space of the second chamber 12, which helps to generate a pressure difference between the first chamber 11 and the second chamber 12, thereby increasing the air flow rate between the first chamber 11 and the second chamber 12, and further enhancing the heat dissipation effect.

In some embodiments, referring to FIG. 3 and FIG. 4, the noise reduction box further includes a partition plate 3. The partition plate 3 is disposed between the first chamber 11 and the second chamber 12 for separating the first chamber 11 and the second chamber 12. The partition plate 3 is provided with a through hole 31, and the first chamber 11 is connected to the second chamber 12 via the through hole 31.

Specifically, the partition plate 3 is disposed between the first chamber 11 and the second chamber 12 for separating the first chamber 11 and the second chamber 12. The partition plate 3 may be a hard alloy plate, a plastic plate, a plastic plate, etc. The partition plate 3 in this embodiment is made through a plastic plus silicone process, which can play a good isolation and sealing role and also help to reduce the overall weight of the noise reduction box. The partition plate 3 is provided with a through hole 31, through which the first chamber 11 is connected to the second chamber 12. The through hole 31 may be a plurality of fine pores, or a long special-shaped hole formed by a plurality of fine pores, which may be specifically designed according to the size of the blower 2 and the noise reduction requirements. The shape of the hole may be circular, square 20) or any irregular shape, which is not limited in this embodiment.

In some embodiments, referring to FIG. 4, in the direction perpendicular to the partition plate 3, the projection of the through hole 31 is located on the periphery of the projection of the air intake 21.

Specifically, the blower 2 is disposed in the middle of the first chamber 11, the air intake 21 of the blower 2 directly faces the second chamber 12. In the direction perpendicular to the partition plate 3, the projection of the through hole 31 is located at the periphery of the projection of the air intake 21. No obstruction exists between the through hole 31 and the air intake 21, so that the air can smoothly enter into the air intake 21.

In some embodiments, referring to FIG. 2, a flexible casing 24 is sheathed around the blower 2.

Specifically, the blower 2 is sleeved with a flexible casing 24 made from materials such as silicone or rubber. The flexible casing 24 has a good impact resistance property and resilience, so that the blower 2 is prevented from being damaged due to bumps during the transportation or installation, that is, providing a better protection for the blower 2. Moreover, the flexible casing 24 also plays a good isolation and sealing role between the blower 2 and the first chamber 11/the second chamber 12, thereby further weakening the transfer of noise and enhancing the noise reduction effect of the noise reduction box.

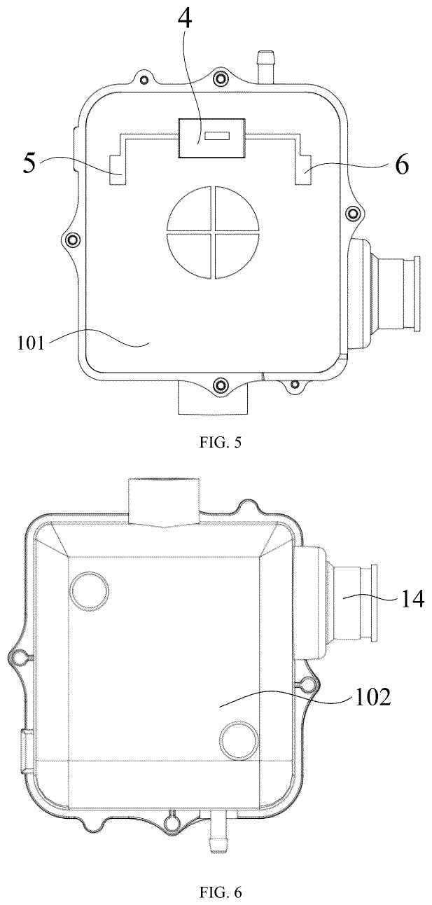

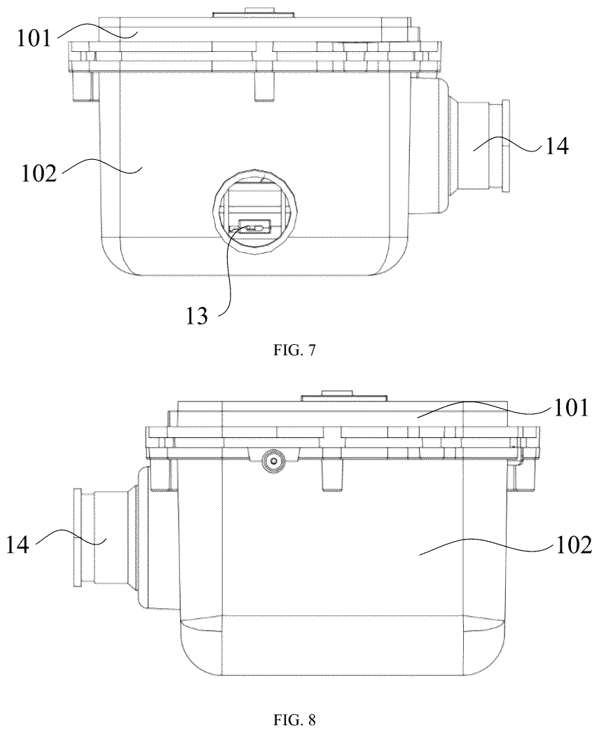

In some embodiments, referring to FIG. 4 and FIG. 5, the noise reduction box also includes a speaker 5. The speaker 5 is fixedly installed on the housing 1, and is used for generating a sound wave to counteract the noise waves of the blower 2.

Specifically, the noise reduction box also includes a speaker 5 that is fixedly installed on the housing 1. A part of the speaker 5 is located in the first chamber 11, and a part of the speaker 5 is exposed outside the housing 1. The speaker 5 can generate a sound wave that has the same frequency as the frequency of the noise of the blower 2 and a phase opposite to the phase of the noise of the blower 2, thereby counteracting the sound wave of the noise and achieving the effect of reducing the noise. It should be noted that in this embodiment, the frequency of the sound wave generated by the speaker 5 is preset according to the specification parameters of the blower 2. The specification parameters of the blower 2 may usually be found in the manual or product introduction of the blower 2.

In some embodiments, referring to FIG. 5, the noise reduction box also includes a control chip 4. The control chip 4 is fixedly installed on the housing 1, and is electrically connected to the speaker 5. The control chip 4 is configured to control the speaker 5 to generate the sound wave.

Specifically, the control chip 4 is fixedly installed outside of the housing 1, and is electrically connected to the speaker 5. Since the control chip 4 is installed outside the housing 1, it is prevented from being affected by the operating temperature inside the housing 1. The control chip 4 is essentially a circuit board integrated with a circuit system. Through the circuit system, the speaker 5 may be controlled to generate the sound wave during the running of the blower 2, and stop generating the sound wave when the blower 2 stops running. In this way, automatic control of the speaker 5 is achieved, without manually turning the speaker on or off, thereby improving the smart design of the noise reduction box and enabling the noise reduction box to have an automatic noise reduction function.

In some embodiments, referring to FIG. 4 and FIG. 5, the noise reduction box also includes a microphone 6. The microphone 6 is fixed installed on the housing 1, and electrically connected to the control chip 4. The microphone 6 is configured to capture the frequency of the noise of the blower 2, and transmit it to the control chip 4.

Specifically, the noise reduction box also includes a microphone 6 that is also electrically connected to the control chip 4, and fixedly installed on the housing 1. A part of the microphone 6 is located in the first chamber 11, and a part thereof is exposed outside the housing 1. The microphone 6 is configured to capture the frequency of the noise of the blower 2, convert the captured frequency of the noise into an electrical signal and transmit it to the control chip 4. After receiving the electrical signal from the microphone 6, the control chip 4 can analyze the frequency of the noise of the blower 2 based on the electrical signal, and control the speaker 5 to generate a sound wave with a frequency same as the frequency of the noise and a phase opposite to the phase of the noise of the blower 2, thereby counteracting the sound wave of the noise. Because of the presence of the microphone 6, the frequency of the sound wave generated by the speaker 5 does not need to be preset. Instead, after the microphone 6 collects the frequency of the noise, the control chip 4 controls the speaker 5 to generate a corresponding sound wave. During a long-term use of the blower 2, the frequency of the noise generated by the blower 2 may be different from the set parameters. In this case, the frequency of the sound wave generated by the speaker 5 may be adjusted with the frequency collected by the microphone 6 so as to match the frequency of the noise of the blower 2, thereby further counteracting effect and improving the noise reduction performance.

In some embodiments, referring to FIG. 4 and FIG. 5, the speaker 5 and the microphone 6 are disposed oppositely on both sides of the first chamber 11.

Specifically, the speaker 5 and the microphone 6 are disposed oppositely on both sides of the first chamber 11. The frequency of the noise is collected on one side, and a sound wave is generated on the other side to counteract the noise frequency. The spatial structure design improves the consistency of sound wave collection and generation, further improving the noise reduction effect of speaker 5 and microphone 6.

An embodiment of the present disclosure also provides a respirator, including any one of the aforementioned noise reduction boxes.

Specifically, the respirator of this embodiment is equipped with any of the aforementioned noise reduction boxes. The noise reduction box will not produce fine particles during the noise reduction process, the risk that the fine particles are inhaled by the patient is eliminated, thereby improving the safety of the respirator. Moreover, the noise reduction box is smaller in volume and occupies less space inside the respirator, which is conducive to the layout of other components in the respirator. It can also reduce the overall weight of the respirator to a certain extent and facilitate the transportation of the respirator.

Finally, it needs to be explained that herein, relational terms such as “first” and “second” are used merely to distinguish an entity or operation from another entity or operation, and do not necessarily require or imply the existence of any such actual relationship or sequence between these entities or operations. Furthermore, the terms “comprising”. “including” or any other variation thereof are intended to cover a non-exclusive inclusion, such that a process, method, article, or terminal device including a list of elements includes not only those elements but also other elements not expressly listed, or elements inherent to the process, method, article or terminal device. Without further limitations, an element defined by the phrase “comprising a . . . ” does not exclude the presence of additional identical elements in the process, method, article or terminal device comprising said element.

The embodiments described above are only preferred embodiments of the present disclosure, and are not intended to limit the present disclosure. Any modifications, equivalent substitutions, improvements, etc, made within the spirit and principles of the present disclosure shall be included in the protection scope of the present disclosure.

1. A noise reduction box, comprising:
a housing and a blower;
wherein the housing comprises a first chamber, a second chamber, an air inlet and an air outlet, wherein the first chamber and the second chamber are connected to each other, and the air inlet and the air outlet are connected to the first chamber respectively;
wherein the blower is disposed in the first chamber, and comprises an air intake connected to the second chamber and a tuyere connected to the air outlet.

2. The noise reduction box according to claim 1, wherein an accommodation space of the first chamber is greater than the accommodation space of the second chamber.

3. The noise reduction box according to claim 1, further comprising a partition plate;
wherein the partition plate is disposed between the first chamber and the second chamber to separate the first chamber and the second chamber;
a through hole is provided on the partition plate, and the first chamber is communicated with the second chamber via the through hole.

4. The noise reduction box according to claim 3, wherein, in a direction perpendicular to the partition plate, a projection of the through hole is located on a periphery of the projection of the air intake.

5. The noise reduction box according to claim 1, wherein a flexible casing is sheathed around the blower.

6. The noise reduction box according to claim 1, further comprising a speaker;
wherein the speaker is fixedly installed on the housing, and the speaker is used for generating a sound wave to counteract a noise wave of the blower.

7. The noise reduction box according to claim 6, further comprising a control chip;
wherein the control chip is fixedly installed on the housing and is electrically connected to the speaker; the control chip is configured to control the speaker to generate the sound wave.

8. The noise reduction box according to claim 7, further comprising a microphone;
wherein the microphone is fixedly installed on the housing, and is electrically connected to the control chip;
wherein the microphone is configured to capture a frequency of the noise of the blower, and transmit the frequency of the noise to the control chip.

9. The noise reduction box according to claim 8, wherein the speaker and the microphone are disposed oppositely on both sides of the first chamber.

10. A respirator comprising:
a noise reduction box, wherein the noise reduction box comprises a housing and a blower;
wherein the housing comprises a first chamber, a second chamber, an air inlet and an air outlet, wherein the first chamber and the second chamber are connected to each other, and the air inlet and the air outlet are connected to the first chamber respectively;
wherein the blower is disposed in the first chamber, and comprises an air intake connected to the second chamber and a tuyere connected to the air outlet.
